SEQUENCE LISTING (1 sequence)

SEQ ID NO: 1 - DNA, 20 bp, Homo sapiens
aggctgctccccccgtggcc

(19) United States
(12) Patent Application Publication
(10) Pub. No.: US 2004/0152097 A1
(43) Pub. Date: Aug. 5, 2004

(54) Gene detection method, detection device, and detection chip

(76) Inventor: Shigeori Takenaka, Koga-shi (JP)

Correspondence Address: COVINGTON & BURLING, ATTN: PATENT DOCKETING, 1201 PENNSYLVANIA AVENUE, N.W., WASHINGTON, DC, 20004-2401, US

(21) Appl. No.: 10/477,718

(22) PCT Filed: Jan. 21, 2002
(86) PCT No.: PCT/JP02/00389

(30) Foreign Application Priority Data
Jan. 19, 2001 (JP) 2001-012413

(51) Int. Cl. C12Q 1/68

(52) U.S. Cl. 435/6

(57) ABSTRACT

The amount in which a probe is immobilized on an electrode can be determined quantitatively, making it possible to quantitatively detect genes with high sensitivity and enhanced throughput. A gene detection method whereby a gene having a specific sequence is hybridized with a probe and electrochemically detected, wherein this method is characterized in that the probe is electrochemically detected.

### TECHNICAL FIELD

The present invention relates to a gene detection method, detection device, and detection chip capable of detecting and analyzing gene base sequences as well as gene abnormalities such as genetic DNA single base substitution SNPs (single nucleotide polymorphisms: mutations of human genetic code), multiple base substitutions, point mutations, and genetic defects.

### BACKGROUND ART

Various gene detection methods of genetic analysis, genetic screening, and the like have been proposed four use in the fields of biology, medicine, and pharmacology. Gene detection is based on a common principle whereby gene fragments (probes) complementary to a target gene are labeled and hybridized with sample gene fragments, the unreacted probe is removed, and a detection reaction is performed using the marker in the probe as an indicator.

Examples of gene detection methods include DNA sequencing, Southern hybridization, fluorescent labeling, and electrochemical detection.

DNA sequencing is a method in which the region with the gene being analyzed is amplified by PCR (polymerase chain reaction), sequencing is performed using fluorescently labeled nucleotides, and the gene sequence of the region is determined.

Southern hybridization commonly entails first fragmenting a sample gene with one or more restriction enzymes and subjecting the gene to gel electrophoresis to achieve size separation. The sample gene is then converted to a single strand and immobilized on a nylon filter or nitrocellulose paper. The single strand thus converted and a complementary single strand (probe) for forming a base pair labeled with a radioactive isotope (RI) are then hybridized, and the filter or nitrocellulose paper is washed. The washed filter or nitrocellulose paper is radiographed and the resulting image is developed to reveal the gene with a specific sequence hybridized with the probe.

There are also techniques in which a sample gene is fluorescently labeled prior to hybridization, the gene and the probe are hybridized, and the fluorescence is measured.

Electrochemical gene detection has also gained prominence recently. These techniques entail electrochemically detecting a double strand hybridized from a gene and a probe, and performing such techniques by using ligands that have electrochemical response and specific binding capacity for double strands is believed to be highly practical because the procedures involved are simple and inexpensive, are carried out using compact equipment, can yield high-sensitivity detection results, and the like. Such electrochemical techniques are particularly promising because of the importance of rapid and high-sensitivity gene detection in high-throughput analysis of gene clusters and in gene expression monitoring.

The above-described gene detection methods are disadvantageous, however, in that it is difficult to uniformly control the amount in which the probe is immobilized on the electrode. Quantitative gene detection is impeded because the amount of probe immobilization is not uniform and varies with the measurement.

It has been proposed to standardize the amount of immobilized probe by determining the amount in which the probe has been immobilized, but the probe amount is still difficult to determine sufficiently accurately with the technologies currently in existence.

For example, the above-described methods of electrochemical gene detection allow hybridized double strands to be detected with high sensitivity but still present difficulties in terms of accurately measuring the amount of immobilized probe or keeping this amount unchanged. As a result, these analysis methods have unacceptable limitations in terms of detection sensitivity.

An object of the present invention, which was perfected in order to overcome the above-described shortcomings of the prior art, is to provide a gene detection method and other means that allow the amount in which a probe is immobilized on an electrode to be quantitatively determined, genes to be quantitatively detected with greater ease and increased sensitivity and throughput, and high-reliability analysis procedures to be carried out.

### DISCLOSURE OF THE INVENTION

The present invention, which is aimed at overcoming the aforementioned shortcomings, provides a gene detection method whereby a gene having a specific sequence is hybridized with a probe and electrochemically detected, wherein this method is characterized in that the probe is electrochemically detected.

“Probe” refers to a probe for analyzing a sample gene. The probe may be a gene having base pair segments that are complementary to the sample gene. Specific examples of such probes include various PCR products having identical or different gene sequences, such as oligonucleotides, mRNA, cDNA, PNA (peptidic nucleic acid), and LNA (Locked Nucleic Acid® from Proligo LLC).

According to the present invention, a probe belonging to a system for gene detection and handling can be quantitatively determined by the electrochemical detection of the probe.

The gene detection method according to the present invention is characterized in that the amount in which the probe is immobilized on an electrode is calculated using such electrochemical detection. This procedure allows genes to be quantitatively detected with high sensitivity.

The present invention, which is aimed at overcoming the aforementioned shortcomings, provides a gene detection method whereby a gene having a specific sequence is hybridized with a probe and electrochemically detected, wherein this method is characterized in that the hybridized double strand is electrochemically detected, and the probe is electrochemically detected as well.

According to the present invention, the double strand and the probe in the system for gene detection and handling can be quantitatively determined by the electrochemical detection of the double strand and the probe.

The gene detection method according to the present invention is characterized in that the amount in which the double strand is produced per unit amount of probe immobilized on the electrode is calculated using such electrochemical detection. This procedure allows genes to be quantitatively detected with high sensitivity by calculating the relative amounts of the immobilized probe and double strand.

The gene detection method according to the present invention is characterized in that an intercalator is introduced into the double strand, and the double strand is electrochemically detected.

The intercalator is a ligand that can enter between the base pairs of a double-strand double helix. Introducing the intercalator makes it possible to identify the double strand with higher sensitivity.

The gene detection method according to the present invention entails electrochemically detecting a gene having a specific sequence, wherein this method comprises a step for fixing a probe to an electrode, a step for electrochemically detecting the amount in which the probe is immobilized on the electrode, a step for hybridizing the probe and the gene to produce a double strand, and a step for introducing an intercalator and electrochemically detecting the double strand.

According to the present method, the amount of the immobilized probe is detected in advance, hybridization is then performed, an intercalator is introduced, and the double strand is subsequently detected. Since the amount of immobilized probe and the amount of double strand can be detected separately, it is possible to accurately determine the amount in which the target gene binds per unit amount of probe and to detect the gene with high sensitivity.

The gene detection method according to the present invention entails electrochemically detecting a gene having a specific sequence, wherein this method comprises a step for fixing a probe to an electrode, a step for electrochemically detecting the amount in which the probe is immobilized on the electrode, a step for reacting the probe and the gene in the presence of an intercalator and hybridizing the probe and the gene to produce a double strand, and a step for introducing the intercalator and electrochemically detecting the double strand.

According to the present method, the amount of the immobilized probe is detected in advance, hybridization is then performed while an intercalator is introduced, and the double strand is subsequently detected. Since the amount of immobilized probe and the amount of double strand can be detected separately, it is possible to accurately determine the amount in which the target gene binds per unit amount of probe and to obtain high-sensitivity quantitative detection results.

The gene detection method according to the present invention entails electrochemically detecting a gene having a specific sequence, wherein this method comprises a step for fixing a probe to an electrode, a step for hybridizing the probe and the gene to produce a double strand, a step for introducing an intercalator into the double strand, and a step for detecting the double strand by an electrochemical measurement and detecting the amount in which the probe is immobilized on the electrode.

According to the present method, probe fixation and hybridization are followed by an operation in which the amount of immobilized probe and the amount of double strand are separately detected at the same time, so the amount in which the double strand is produced per unit amount of immobilized probe can be determined with high accuracy and the two electrochemical measurements can be performed in a single session, simplifying and accelerating the detection procedure.

The gene detection method according to the present invention entails electrochemically detecting a gene having a specific sequence, wherein this method comprises a step for fixing a probe to an electrode, a step for reacting the probe and the gene in the presence of an intercalator and hybridizing the probe and the gene to produce a double strand, and a step for detecting the double strand by an electrochemical measurement and detecting the amount of the probe immobilized on the electrode.

The present method allows the detection procedure to be simplified and accelerated because the amounts of the immobilized probe and double strand can be quantitatively detected at the same time.

The gene detection method according to the present invention entails electrochemically detecting a gene having a specific sequence, wherein this method comprises a step for hybridizing a probe and a gene to produce a double strand, a step for introducing an intercalator into the double strand, a step for fixing the probe to an electrode, and a step for detecting the double strand by an electrochemical measurement and for detecting the amount of the probe immobilized on the electrode.

According to the present method, a probe and a sample gene are hybridized in a uniform solution or in a solution in the vicinity of an electrode surface, an intercalator is introduced thereinto for labeling, the probe is then immobilized on the electrode, and the amount of immobilized probe and the amount of double strand are separately detected at the same time, making it possible to accurately determine the amount in which the double strand is produced per unit amount of immobilized probe. An advantage of the present method is that reactions can be conducted with higher efficiency because the hybridization and intercalation are carried out in a solution without immobilizing the probe on the electrode.

The gene detection method according to the present invention entails electrochemically detecting a gene having a specific sequence, wherein this method comprises a step for reacting the probe and the gene in the presence of an intercalator and hybridizing the probe and the gene to produce a double strand, a step for fixing the probe in the double strand to an electrode, and a step for detecting the double strand by an electrochemical measurement and for detecting the amount in which the probe is immobilized on the electrode.

An advantage of the present method is that reaction efficiency can be increased because the amount in which the double strand is produced per unit amount of immobilized probe can be determined with high accuracy, and the components can be reacted in solution.

The gene detection method is characterized in that the probe has an electrochemical signal section and that the amount in which the probe is immobilized on the electrode is detected by measuring the electric current flowing through the electrochemical signal section.

The present method allows the amount of immobilized probe to be quantitatively detected with high accuracy with the aid of an electrochemical signal section by providing the probe with such a section. The term “electrochemical signal section of a probe” refers to a site in the probe that has been chemically modified with a substance that exhibits electrochemical response. Examples of substances that exhibit such electrochemical response include substances having redox activity, substances having oxidation activity, and substances having reduction activity.

The gene detection method is characterized in that the probe has an electrode immobilization section at one end thereof, and an electrochemical signal section at the opposite end.

The gene detection method is characterized in that the electrochemical signal section of the probe and the double strand have different detection potentials. Although the two detection potentials can each be measured after being set to the same level, using two different detection potentials has the advantage of allowing the two detection potentials to be measured at the same time. Such concurrent measurement results in a simplified and accelerated detection procedure.

The electrochemical signal section may be composed of anthraquinone, ferrocene, catecholamine, a metal bipyridine, a metal phenanthrine complex, viologen, or the like.

The intercalator should preferably be a threading intercalator. A threading intercalator is a reagent that forms a complex in which two or three substituents extend into the major and minor grooves when intercalated into the double strand. Consequently, one of the substituents functions as a stopper when the threading intercalator separates from the double strand. This approach is advantageous in that dissociation from the nucleic acids of the double strand is slowed down considerably, and the double strand is stabilized. A ferrocene-modified threading intercalator is particularly preferred.

The gene detection device of the present invention is a device whereby a gene having a specific sequence is hybridized with a probe and electrochemically detected, wherein this device comprises means for electrochemically detecting the hybridized double strand, and means for electrochemically detecting the probe.

The gene detection chip of the present invention comprises an electrode with an immobilized probe for hybridizing a gene having a specific sequence, and a common electrode that functions as a counter electrode for the first electrode, wherein this gene detection chip is characterized in that voltage is applied between the electrode and the common electrode to allow the hybridized double strand to be electrochemically detected and to allow the probe to be electrochemically detected as well. The chip may, for example, be used to detect gene expression levels, base sequences, single base substitution SNPs, multiple base substitutions, point mutations, translocations, defects, amplifications, and triplet repeats.

The electrode of the inventive gene detection chip may also be composed of a plurality of pin electrodes.

The detection chip may also be used for genetic screening. Gene expression levels, base sequences, and other factors related to monogenic disorders (such as muscular dystrophy, hemophilia, and phenyl ketonuria) and multifactorial genetic diseases (such as diabetes, cancer, hypertension, myocardial infarction, and obesity) can be diagnosed, or premorbid gene expression levels, base sequences, and other factors can be diagnosed by genetic screening based on the use of the inventive detection chip, which can thus be employed as a diagnostic material for selecting an appropriate treatment or drug.

### BRIEF DESCRIPTION OF THE DRAWINGS

FIG. 1 is a diagram schematically depicting the gene detection method pertaining to the present invention;

FIG. 2 is a perspective view illustrating the overall structure of the gene detection device pertaining to the present invention;

FIG. 3 is a perspective view illustrating the overall structure of the gene detection chip pertaining to the present invention;

FIG. 4 is a measurement result illustrating the manner in which detection current varies in Example 1;

FIG. 5 is a measurement result illustrating the manner in which detection current varies in Example 1;

FIG. 6 is a graph illustrating the manner in which the Ib/Ia value varies in Example 1;

FIG. 7 is a measurement result illustrating the manner in which detection current varies in Example 2; and

FIG. 8 is a measurement result illustrating the manner in which detection current varies in Example 3.

### BEST MODE FOR CARRYING OUT THE INVENTION

Examples of the gene detection method, detection device, and detection chip pertaining to the present invention will now be described with reference to the accompanying drawings. The drawings merely illustrate examples of the present invention and are nonlimiting in nature.

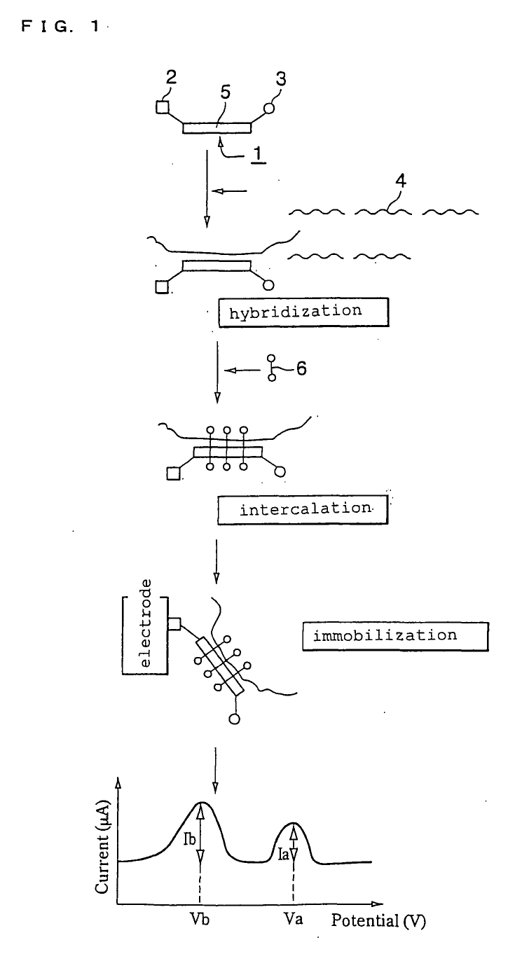

FIG. 1 is a diagram schematically depicting the gene detection method pertaining to the present invention. In FIG. 1, the probe 1 comprises a probe main body 5, an electrode immobilization section 2 at one end of the probe main body 5, and an electrochemical signal section 3 at the other end. A sample gene 4 and the probe 1 are first hybridized in a solution, whereupon the probe main body 5 and the sample gene 4 having a matching base sequence bind to each other and form a double strand. At this time, adding an intercalator 6 initiates intercalation and causes the intercalator 6 to bind to the double strand. The probe 1 is then immobilized on an electrode, and an electrochemical measurement is performed by cyclic voltammetry (CV), differential pulse voltammetry (DPV), or the like. The detection potential Va on the electrochemical signal section 3 and the detection potential Vb on the intercalator are set to different levels, the electric currents are detected, and values Ia and Ib are calculated by subtracting base lines from the electric currents of the corresponding detected peaks. The Ib and Ia ratio (Ib/Ia) expresses the amount in which the double strand is produced per unit amount of immobilized probe.

Although the above example was described with reference to a detection method in which the intercalation reaction was performed in a solution, the present invention can also be applied to cases in which a different sequence is adopted to perform the steps for immobilizing the probe on an electrode, detecting the amount of immobilized probe, introducing the intercalator, and the like.

Any electrode can be used in the present invention as long as a probe can be immobilized on this electrode. Preferred examples include gold, glassy carbon, and carbon.

Any substance having electrochemical activity can be used for the electrochemical signal section of the probe used in the present invention, with a substance having redox activity being particularly preferred. Preferred examples of substances having such redox activity include anthraquinone, ferrocene, catecholamine, metal bipyridine, metal phenanthrine complexes, and viologen, of which ferrocene is particularly preferred.

The probe can be any chemically synthesized DNA or gene obtained by a process in which a gene extracted from a biological sample is cut with a restriction enzyme and purified by electrophoretic separation or the like. The probe sequence should preferably be preset. Any known technique may be used for setting the probe sequence.

The electrochemical signal section can be introduced into the probe by a method in which a substance exhibiting electrochemical response is caused by amide linkage to bind to the 5′ end and/or 3′ end of the probe main body. It is also possible to cause a substance that exhibits electrochemical response to bind to the 5′ end and/or 3′ end by amide linkage via a linker. The probe main body and the substance that exhibits electrochemical response may be bound together by a common method. Following is a description of an example in which a probe is fabricated by the binding of ferrocene and a probe main body obtained by introducing amino groups into the 5′ end. The probe is dissolved in an appropriate buffer (such as a sodium carbonate/sodium bicarbonate buffer), an organic solvent (such as DMSO) containing, for example, ferrocenecarboxylic acid N-hydroxysuccinimide ester is added, a reaction is carried out, and the product is purified by HPLC or the like, yielding a probe in which the ferrocene is bound to the 5′ end.

The probe can be immobilized on the electrode by a common method. When, for example, the electrode is made of gold, a thiol group (SH group) is introduced into the probe main body, and the probe is caused to bind to the electrode by the gold-sulfur coordinated linkage between the gold and sulfur. Methods for introducing thiol groups into the probe main body are described by Mizuo MAEDA, Koji NAKANO, Shinji UCHIDA, and Makoto TAKAGI in Chemistry Letters, 1805-1808 (1994) and by B. A. Connolly in Nucleic Acids Rs., 13, 4484 (1985). A probe provided with thiol groups by the above-described methods is added in drops to a gold electrode and is allowed to stand for several hours at a low temperature (for example, 4° C.), whereby the ferrocene-modified probe is immobilized on the gold electrode.

According to another method, a carboxylic acid is introduced into an electrode surface by oxidizing glassy carbon with potassium permanganate, and modified amino groups and amide bonds are formed on the probe, making immobilization possible. A process for immobilizing materials to glassy carbon is described by Kelly M. Millan and Susan R. Mikkelsen in Analitical Chemistry, 65, 2317-2323 (1993).

SH-gold bonding and pretreatment methods that precede the gold plating of electrode surfaces are described, for example, by C. D. Bain in J. Am. Chem. Soc. (No. 111, p. 321, 1989) and by J. J. Gooding in Anal Chem. (No. 70, p. 2396, 1998).

The electrode with the bound probe is introduced into a sample solution containing a sample gene, whereby the sample gene (whose sequence is complementary to that of the probe) is hybridized and a double strand is formed. A known hybridization method can be used.

Examples of intercalators suitable for the present invention include ferrocene, catecholamine, metal bipyridine complex, metal phenanthrine complex, viologen, and materials whose active ingredient is a threading intercalator into which the above compounds have been introduced. Of these, ferrocene is particularly preferred as the threading intercalator. The following active ingredients may also be used: ethidium, ethidium bromide, acridine, aminoacridine, acridine orange, Proflavine, Ellipticine, Actinomycin D, Daunomycin, Mitomycin C, tris(phenanthroline) zinc complex, tris(phenanthroline) ruthenium complex, tris(phenanthroline) cobalt complex, di(phenanthroline) zinc complex, di(phenanthroline) ruthenium complex, di(phenanthroline) cobalt complex, bipyridine platinum complex, terpyridine platinum complex, phenanthroline platinum complex, tris(bipyridine) zinc complex, tris(bipyridine) ruthenium complex, tris(bipyridine) cobalt complex, di(bipyridine) zinc complex, di(bipyridine) ruthenium complex, and di(bipyridine)cobalt complex.

The intercalator is used in a concentration of several nM to several mM during the intercalation reaction. A concentration of between 0.1 mM and 5 mM is preferred, and a concentration of 0.5 mM is particularly preferred.

The intercalator is allowed to enter the space between the layers of the double strand, yielding a charge-transfer complex. The electric current flowing through the electrode varies with the intercalator. The electric current is generated by the redox reaction of the intercalator bound to the double strand. The extent to which the double strand has been formed can be quantitatively detected as the degree of intercalation. Consequently, the amount of the double strand can be measured by detecting the value of the electric current.

By contrast, the amount in which the probe is immobilized on the electrode can be quantitatively detected based on the variation in the electric current due to the redox reaction involving the electrochemical signal section of the probe.

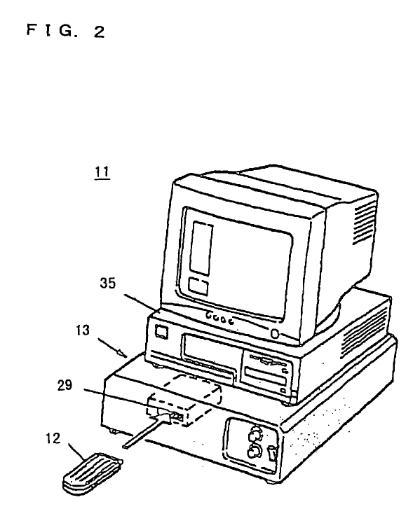

FIG. 2 is a perspective view illustrating the overall structure of the detection device pertaining to the present invention. In FIG. 2, the gene detection device 11 pertaining to the present invention comprises detection chip 12, a personal computer 35, and a measurement device 13. This device has an insertion slot 29 capable of accommodating the detection chip 12 and has the ability to electrochemically detect the probe and the double strand produced by hybridization.

Cyclic voltammograms, differential pulse voltammograms, potentiostats, and the like may be used as the electrochemical detection means.

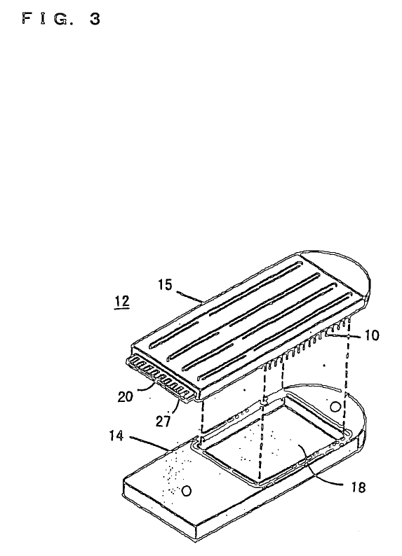

FIG. 3 is a diagram depicting the structure of the detection chip 12. The detection chip 12 comprises a frame 14 provided with a centrally located depression 18, and a main body 15 detachably mounted on the frame 14, as shown in FIG. 3. The depression 18 can be filled with a solution (probe, sample gene, intercalator, washing solution, or the like). A large number of pin electrodes 10 are uniformly arranged in the area of the main body 15 that corresponds to the depression 18 in the frame 14. The depression 18 is sequentially filled and washed with specific solutions; hybridization, intercalation, and the like are performed; the detection chip 12 is then introduced into the insertion slot 29; terminals 27 for a common electrode and terminals 20 for the pin electrodes are connected to the receiving terminals of the measurement device and are connected to a voltage circuit by a selection switch; and low voltage is applied between the common electrode and the pin electrodes 10, whereupon a weak electric current is caused to flow through the voltage circuit and the common electrode between the pin electrodes 10 and the intercalator-labeled portions and/or chemically signaled portions of the probe. The gene is detected by detecting this electric current.

### EXAMPLES

Examples will now be described, but the present invention is not limited by these examples.

### Example 1

An SNP detection experiment was performed involving a G→C transversion in the p53 human gene at codon 72 and exon 4.

First, the following probe was immobilized on a gold electrode.

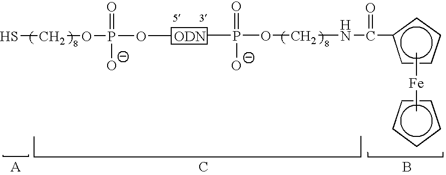

The equation shows that the probe comprised a probe main body C having —(CH₂)₈— as linkers at both ends of an oligonucleotide (ODN), an electrode immobilizing section A composed of thiol groups attached to the 5′ end thereof, and an electrochemical signal section B with ferrocene at the 3′ end. The probe main body C is given by Eq. (a) below.

5′-AGGCTGCTCCCCCCGTGGCC-3′ (a)

The probe was bound and immobilized on the gold electrode, a PCR product (280 bp) was thermally denatured as the target DNA, and a hybridization reaction was carried out. The unbound target DNA was removed, and an intercalator composed of the ferrocene-modified naphthalene diimide shown below was added and caused to bind to the double strand section of the DNA.

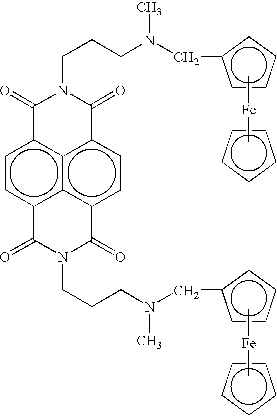

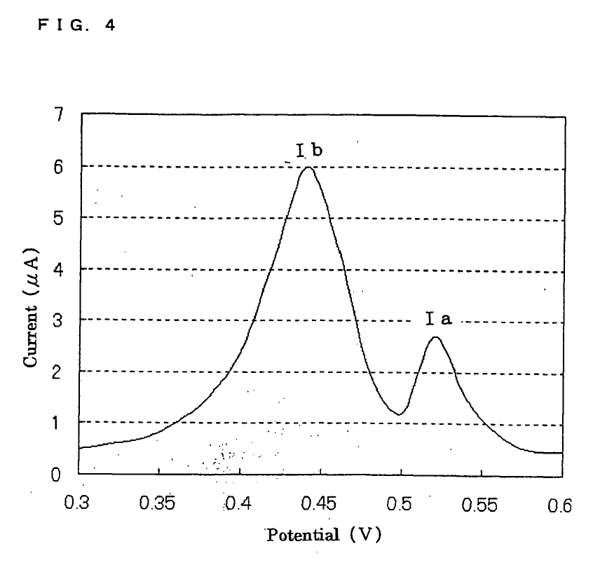

A DPV (differential pulse voltammetry) measurement was then performed in an electrolyte measurement solution (0.1M AcOH-AcOK (pH: 5.6), 0.1M KCl, 0.05 mM NFc). The results are shown in FIG. 4.

An intercalator-derived signal (Ib) was detected at a potential of 0.44 V (Ag/AgCl reference electrode), and a probe-derived signal (Ia) was detected at a potential of 0.52 V (Ag/AgCl reference electrode), as shown in FIG. 4.

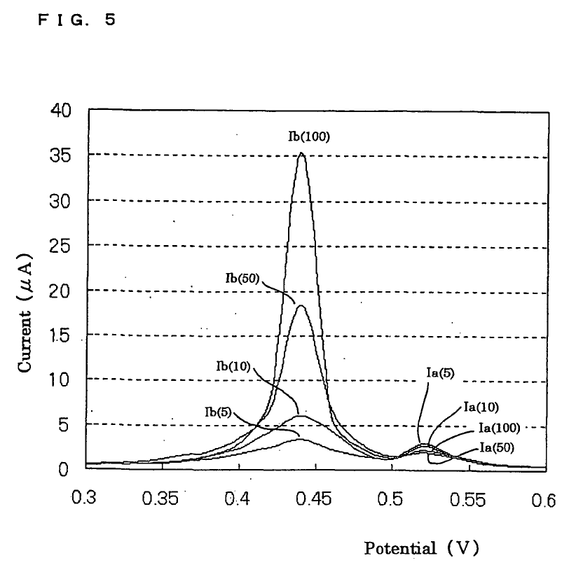

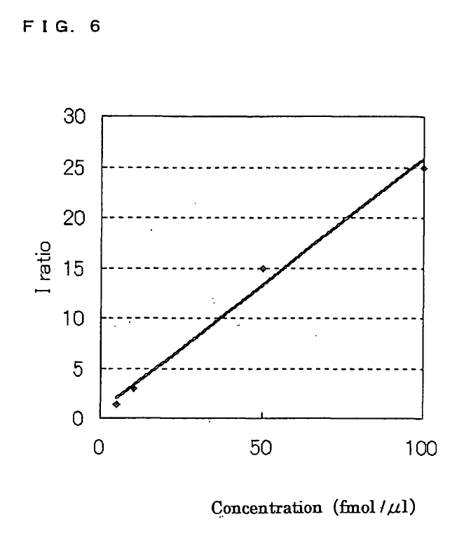

FIG. 5 depicts results obtained when the same experiment was conducted by varying the target DNA concentration (x) (5, 10, 50, 100 fmol/μl). FIG. 6 depicts results obtained when the values Ia (5) to Ia (100) and Ib (5) to Ib (100) were obtained by subtracting the baseline for the peak current values detected in each case, the ratios thereof were calculated according to Eq. (1) below, and the calculated ratios were plotted.

I ratio (x)=Ib(x)/Ia(x) Eq. (1)

The amount Ia(x) in which the probe is immobilized on the electrode varies with the electrode, as can be seen in FIG. 6. A risk therefore exists that merely detecting the amount Ib(x) of double strand DNA will create an error when the amounts of double strand DNA per unit of probe amount are calculated for different electrodes. In the present example, however, the formation of double strand DNA could be accurately quantified by initially correcting the probe amount according to Eq. (1).

### Example 2

An experiment was performed in the same manner as in Example 1 except that the probe used in Example 1 above was replaced with the probe shown below.

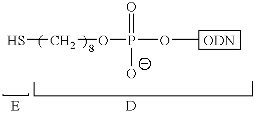

The equation shows that the probe comprised a probe main body D having —(CH₂)₈— as linkers at one end of an oligonucleotide (ODN), and a section E as an electrode immobilization section and an electrochemical signal section composed of thiol groups attached to the 5′ end thereof. Section E simultaneously functioned as an electrode immobilization section and an electrochemical signal section. Electric current variations, based on the cleavage processes that accompanied the oxidation of S in the Au—S bonds resulting from the bonding of section E to the gold electrode, were detected as electrochemical signals. The probe main body D was SNP Pro72 of the p53 gene.

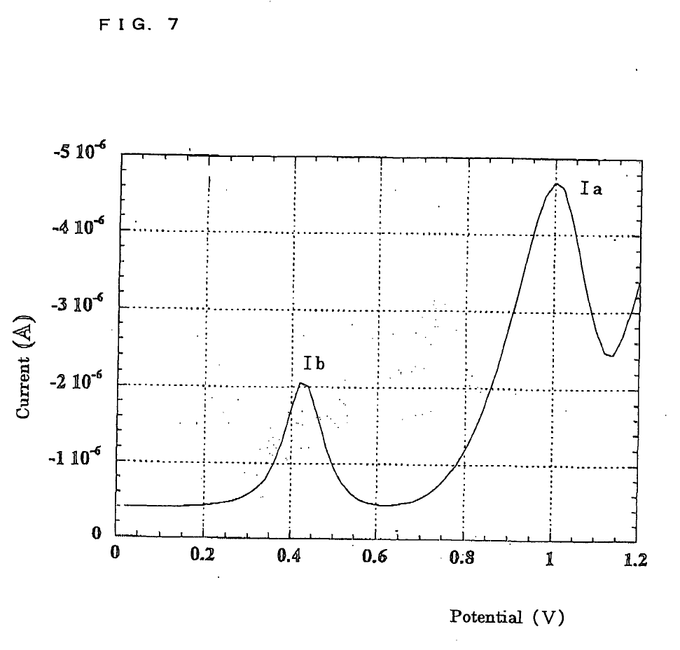

The probe was bound and immobilized on a gold electrode, a PCR product (280 bp) was thermally denatured as the target DNA, and a hybridization reaction was carried out. The unbound target DNA was removed, an intercalator was added, and a DPV (differential pulse voltammetry) measurement was performed in an electrolyte measurement solution (0.1M AcOH-AcOK (pH: 5.6), 0.1M KCl, 0.05 mM NFc). The results are shown in FIG. 7.

An intercalator-derived signal (Ib) was detected at a potential of 0.43 V (Ag/AgCl reference electrode), and a probe-derived signal (Ia) was detected at a potential of 1.00 V (Ag/AgCl reference electrode), as shown in FIG. 7.

An experiment was conducted in the same manner as in Example 1 by varying the amount of target DNA, and it was confirmed that a quantitative determination could be made.

### Example 3

An experiment was performed in the same manner as in Example 1 except that the probe used in Example 1 above was replaced with the probe shown below.

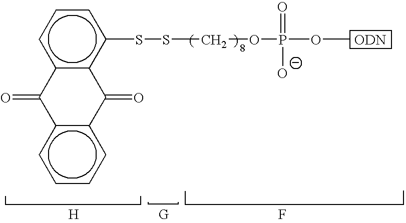

The equation shows that the probe comprised a probe main body F having —(CH₂)₆— as linkers at one end of an oligonucleotide (ODN), an electrode immobilization section G composed of disulfide bonds at the 5′ end thereof, and an electrochemical signal section H composed of anthraquinone. The probe main body F was SNP Pro72 of the p53 gene.

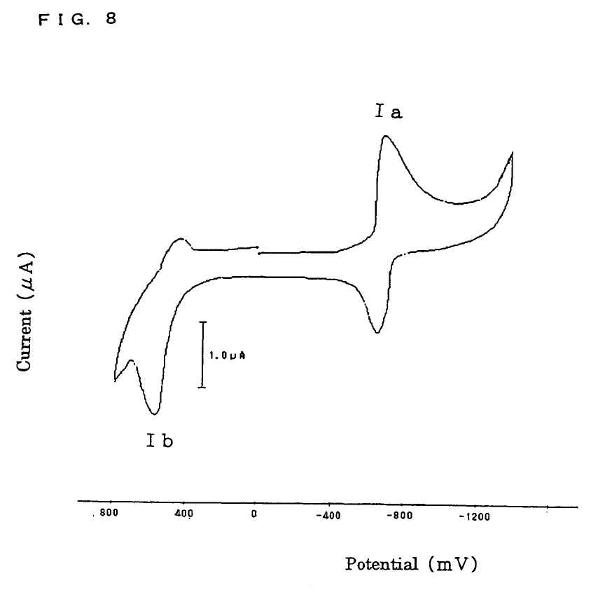

The probe was bound and immobilized on a gold electrode, a PCR product (280 bp) was thermally denatured as the target DNA, and a hybridization reaction was carried out. The unbound target DNA was removed, an intercalator was added, and a CV (cyclic voltammetry) measurement was performed in an electrolyte measurement solution (0.1M AcOH-AcOK (pH: 5.6), 0.1M KCl, 0.05mM NFc). The results are shown in FIG. 8.

An intercalator-derived signal (Ib) was detected at a CV oxidation potential of 500 mV, as shown in FIG. 8. A signal (Ia) derived from the electrochemical signal section H of the probe was detected at a reduction potential of 700 mV. The target DNA concentration (x) was measured and the Ib(x)/Ia(x) ratio calculated in the same manner as in Example 1, and it was confirmed that a quantitative determination could be made.

### Example 4

The same probe as the one used in Example 3 above was hybridized with a sample DNA without being immobilized on an electrode. The gold electrode was immersed in the solution to bind the probe to the surface of the gold electrode via the electrode immobilization section G. An intercalator was added to the solution and bound to the double strand DNA, and a CV (cyclic voltammetry) measurement was performed in an electrolyte measurement solution (0.1M AcOH-AcOK (pH: 5.6), 0.1M KCl, 0.05 mM NFc).

As a result, it was learned that in comparison with Examples 1-3, in which the probe DNA had been immobilized on the electrode in advance, the detection current (Ib) occurring at the oxidation potential of 500 mV and the detection current (Ia) occurring at the reduction potential of 700 mV had decreased in magnitude by about 70%, and a quantitative determination could successfully be made using the I ratio (Ib/Ia).

### INDUSTRIAL APPLICABILITY

The gene detection method, detection device, and detection chip pertaining to the present invention allow the amount in which a probe is immobilized on an electrode to be determined quantitatively, making it possible to quantitatively determine/analyze genes with improved sensitivity, high throughput, and greater convenience.

The high-sensitivity and high-throughput detection device of the present invention is an efficient means of analyzing the relations between genes and their expression in the biological and medical fields. Genetic screening can also be performed by analyzing drug-metabolizing enzymes, cancer-suppressing genes, and other specific genes with the aid of the inventive detection/analysis device for determining gene expression levels, base sequences, single base substitution SNPs, multiple base substitutions, point mutations, translocations, defects, amplifications, and triplet repeats.

For example, the detection device pertaining to the present invention performs high-sensitivity, high-throughput procedures, making it possible to collect genetic data for Japanese individuals, to identify genes associated with certain illnesses, and to predict/prevent diseases in the future.

Genetic screening can be useful for selecting the right treatment or picking drugs with minimal side effects.

In addition, results from the genetic analysis of a disease can be used to develop drugs without performing repeated clinical trials or the like.

1. A gene detection method, wherein the method is for electrochemically detecting a gene having a specific sequence by hybridizing said gene with a probe, and wherein the probe is electrochemically detected.

2. The gene detection method according to claim 1, wherein the amount in which the probe is immobilized on an electrode is calculated using the electrochemical detection.

3. A gene detection method, wherein the method is for electrochemically detecting a gene having a specific sequence by hybridizing the gene with a probe, and wherein the hybridized double strand is electrochemically detected, and the probe is also electrochemically detected.

4. The gene detection method according to claim 1, wherein the amount of the double strand produced per unit amount of probe immobilized on the electrode is calculated using the electrochemical detection.

5. The gene detection method according to claim 3, wherein an intercalator is introduced into the double strand, and the double strand is electrochemically detected.

6. A gene detection method for electrochemically detecting a gene having a specific sequence, comprising the steps of:
immobilizing a probe on an electrode;
electrochemically detecting the amount in which the probe is immobilized on the electrode;
hybridizing the probe and the gene to produce a double strand; and
introducing an intercalator and electrochemically detecting the double strand.

7. A gene detection method for electrochemically detecting a gene having a specific sequence, comprising the steps of:
immobilizing a probe on an electrode;
reacting the probe and the gene in the presence of an intercalator and hybridizing the probe and the gene to produce a double strand; and
introducing the intercalator and electrochemically detecting the double strand.

8. A gene detection method for electrochemically detecting a gene having a specific sequence, comprising the steps of:
immobilizing a probe on an electrode;
hybridizing the probe and the gene to produce a double strand;
introducing an intercalator into the double strand; and
detecting the double strand by an electrochemical measurement and detecting the amount in which the probe is immobilized on the electrode.

9. A gene detection method for electrochemically detecting a gene having a specific sequence, comprising the steps of:
immobilizing a probe on an electrode;
reacting the probe and the gene in the presence of an intercalator and hybridizing the probe and the gene to produce a double strand; and
detecting the double strand by an electrochemical measurement and detecting the amount in which the probe is immobilized on the electrode.

10. A gene detection method for electrochemically detecting a gene having a specific sequence, comprising the steps of:
hybridizing a probe and said gene to produce a double strand;
introducing an intercalator into the double strand;
immobilizing the probe on an electrode; and
detecting the double strand by an electrochemical measurement and detecting the amount in which the probe is immobilized on the electrode.

11. A gene detection method for electrochemically detecting a gene having a specific sequence, comprising the steps of:
reacting a probe and said gene in the presence of an intercalator and hybridizing the probe and the gene to produce a double strand;
immobilizing the probe in the double strand on an electrode; and
detecting the double strand by an electrochemical measurement and detecting the amount in which the probe is immobilized on the electrode.

12. The gene detection method according to claim 1, wherein the probe has an electrochemical signal section and the amount in which the probe is immobilized on the electrode is detected by measuring the electric current flowing through the electrochemical signal section.

13. The gene detection method according to claim 12, wherein the probe has an electrode immobilization section at one end thereof, and an electrochemical signal section at the opposite end.

14. The gene detection method according to claim 12, wherein the electrochemical signal section of the probe and the double strand have different detection potentials.

15. The gene detection method according to claim 12, wherein the electrochemical signal section is anthraquinone, ferrocene, catecholamine, a metal bipyridine, a metal phenanthrine complex, viologen, or a thiol group.

16. The gene detection method according to claim 5, wherein the intercalator is a threading intercalator.

17. A gene detection device, wherein the device is for detecting a gene having a specific sequence electrochemically by hybridizing the gene with a probe, and comprises:
means for electrochemically detecting the hybridized double strand; and
means for electrochemically detecting the probe.

18. A gene detection chip, wherein the gene detection chip comprises an electrode to which a first probe to be hybridized with a gene having a specific sequence is immobilized, and a common electrode that functions as a counter electrode for the first electrode, and the gene detection chip is capable of electrochemically detecting the hybridized double strand and also capable of electrochemically detecting the probe by applying a voltage between the first electrode and the common electrode.

19. The gene detection chip according to claim 18, wherein the first electrode is composed of a plurality of pin electrodes.
